(19) United States
(12) Patent Application Publication
(10) Pub. No.: US 2007/0161808 A1
(43) Pub. Date: Jul. 12, 2007

(54) Process for the preparation of aq4n

(75) Inventors: Ian Timothy William Matthews, Horsham (GB); Ronald Michael Scott, Craigavon (GB); John Francis Barry, Craigavon (GB); Stephen William Hughes, Craigavon (GB); Ann Heslip, Craigavon (GB)

Correspondence Address: MICHAEL BEST & FRIEDRICH, LLP, ONE SOUTH PINCKNEY STREET, P O BOX 1806, MADISON, WI, 53701, US

(73) Assignee: KUDOS PHARMACEUTICALS LIMITED, MILTON ROAD (GB)

(21) Appl. No.: 10/597,865

(22) PCT Filed: Feb. 11, 2005
(86) PCT No.: PCT/GB05/00496

§ 371 Date: Aug. 10, 2006

Related U.S. Application Data
(60) Provisional application No. 60/544,778, filed on Feb. 13, 2004.

(51) Int. Cl. C07C 225/26

(52) U.S. Cl. 552/238

(57) ABSTRACT

A process for the preparation of compound AQ4N of formula (2) or a salt or solvate thereof wherein the said process includes the reaction step: Formula (1), Formula (2), where compound AQ4 of formula (1) is oxidised to compound AQ4N of formula (2) with an oxidising agent at a reaction temperature not exceeding 10° C.

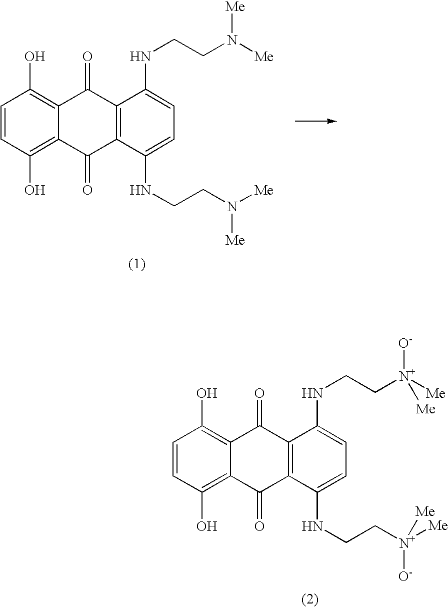

The present invention relates to a process for the preparation of AQ4N, and its salts and solvates. In particular, the process can be used on an industrial scale and is suitable for the preparation of pharmaceutically pure compounds.

### BACKGROUND TO THE INVENTION

AQ4N is a non-toxic prodrug that has use in the treatment of cancer. The active drug is the cytotoxic compound AQ4, which is produced in vivo from AQ4N by reductive metabolism in hypoxic cells. This process is the reverse of the oxidation step used in the synthesis of AQ4N from AQ4.

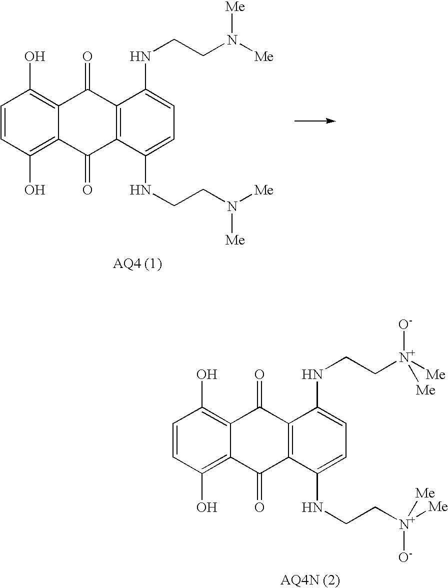

It is often convenient or desirable to prepare, purify, and/or handle a corresponding solvate or salt, such as a pharmaceutically-acceptable salt, of AQ4N. Examples of pharmaceutically acceptable salts are discussed in Berge et al., 1977, “Pharmaceutically Acceptable Salts,” J. Pharm. Sci., Vol. 66, pp. 1-19. AQ4N has been reported in the form of many salts or solvates (J. Chem. Soc., Perkin Trans. I, 1999, 2755; WO 00/05194; WO 03/078387).

However, a problem with known preparations of AQ4N and salts or solvates thereof is that the resulting products contain, in varying degrees, an impurity known as AQMN. AQMN or 1-amino-4-{[2-(dimethylamino)ethyl]amino}-5,8-dihydroxyanthraquinone is formed by degradation of AQ4N. AQMN is an undesirable contaminant in a compound that is intended to be administered as a non-toxic prodrug.

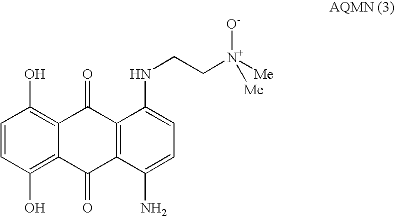

AQ4N and salts or solvates thereof may be prepared using a process that employs several reaction steps, as detailed in WO 00/05194 and set out below. This method was carried out on a small scale (˜0.1 mol). One step in this process is the conversion of 3,6-difluorophthalic anhydride (DFPA) and p-hydroquinone into 1, 4-difluoro-5,8-dihyroxyanthracene (DDA) by a Friedel-Crafts acylation using an aluminium chloride catalyst.

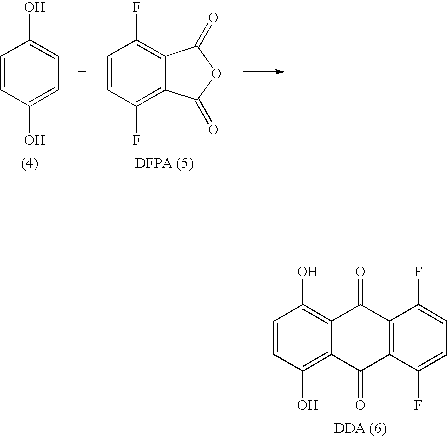

The reaction was performed by heating a powdered mixture of the solids (4), (5), sodium chloride and aluminium trichloride to a temperature of 220° C.

The fluorine atoms in DDA are then substituted by N,N-dimethylethylenediamine in the following reaction to produce AQ4.

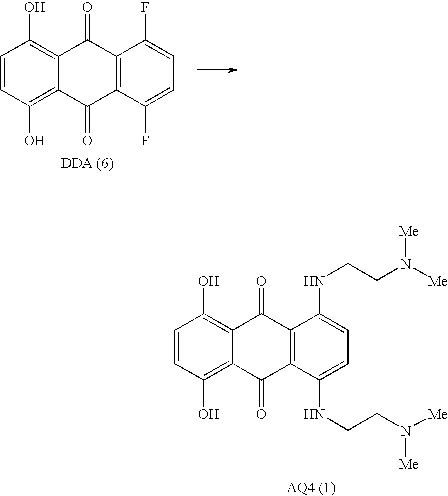

In the method described in WO 00/05194, this reaction step produced only moderate yields of product (˜40%), even on laboratory sized scale of about 0.1 moles.

The next reaction step is then performed to oxidise AQ4 into AQ4N.

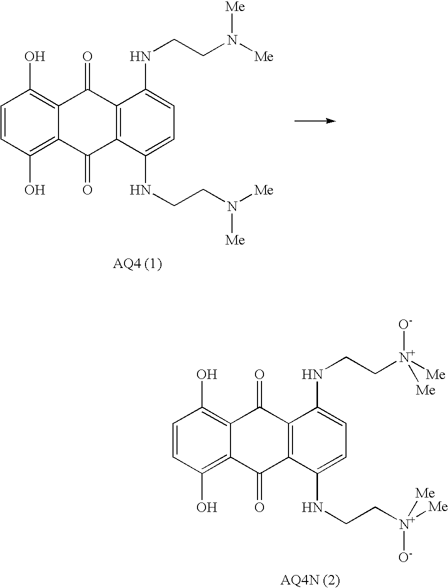

The method described in WO 00/05194 uses the Davis reagent (2-benzene-sulfonyl-3-phenyl-oxaziridine) as the oxidising agent and the reaction is performed at room temperature.

It is possible to isolate AQ4N at this point or further convert it, in another reaction step, to a salt or solvate. The inorganic dihydrochloride salt has been prepared on a laboratory scale by treating a methanolic solution of crude AQ4N, at room temperature, with anhydrous HCl gas (WO 00/05194). Organic acid salts of AQ4N have also been prepared by the addition of a methanolic solution containing the organic acid (WO 03/078387).

### DISCLOSURE OF THE INVENTION

The present invention discloses an improved process for the preparation of AQ4N and salts or solvates thereof. This process can be carried out on an industrial scale (for example, at least 0.2 mol) and includes improved methods for the synthesis of intermediate compounds. These improved methods aim to result in a reduced level of contaminants in the final product which may be pharmaceutically pure. The process of the invention, in general, uses the reaction sequence and intermediates described above.

Oxidation of AQ4 to AQ4N

A problem with the conversion of AQ4 into AQ4N is that incomplete oxidation of AQ4 can lead to the formation of a partially oxidised bi-product AQ4M (7). However, harsh conditions employed to ensure complete oxidation often result in the degradation of AQ4N into AQMN. The degradant AQMN is obtained even under relatively mild conditions (WO 03/078387). This degradative pathway reduces both the yield and purity of the final product.

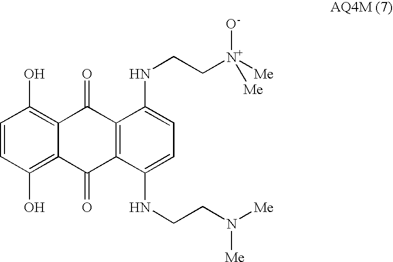

An aspect of the invention is to perform the oxidation of AQ4 to AQ4N at a temperature not exceeding 10° C. The reaction temperature is preferably less than 5° C., more preferably less than 0° C. and possibly the reaction temperature does not exceed −7° C. The reaction would normally be carried out at a temperature higher than −20° C. Addition of the oxidant to the reaction mixture is carried out when the reaction solution is at a temperature not exceeding 0° C., more preferably less than −7° C. and even more preferable is a reaction solution temperature not exceeding −10° C. An appropriate solvent for the reaction temperature should be chosen.

An oxidant that selectively oxidises AQ4 to AQ4N is preferably used in this aspect of the invention. Suitable oxidising agents include hydrogen peroxide, oxaziridines or peracids or salts of peracids, such as m-chloroperbenzoic acid, perbenzoic acid, peracetic and magnesium monoperoxyphthalate. The oxidising agent is preferably hydrogen peroxide or more preferably magnesium monoperoxyphthalate. The oxidant magnesium monoperoxyphthalate is stable in air and soluble in water, making it more manageable when used on a large scale. The solvent used should be compatible with the chosen oxidising agent.

Suitable solvents for the oxidation of AQ4 to AQ4N include dichloromethane, chloroform, dichloroethanes, carbon tetrachloride, toluene, 1,2-propanediol or a solvent mixture of any combination of these solvents. All these solvents can also be used as a mixture with or without aliphatic alkyl alcohols. The reaction solvent is preferably 1,2-propanediol or more preferably a solvent mix of dichloromethane and an aliphatic alkyl alcohol.

Accordingly, a preferred embodiment uses magnesium monoperoxyphthlate. Addition of magnesium monoperoxyphthalate is carried out a temperature of between −15° C. and −5° C., more preferably at about −11° C. After addition of the oxidant, the reaction is allowed to stir at a temperature of between −15° C. and 5° C., more preferably about 0° C. The preferred solvent for this reaction is a mixture of methanol and dichloromethane, preferably in a volume ratio of between 1:1.5 and 1:2.5.

Formation of Salts of AQ4N

In the previous method, the dihydrochloride salt of AQ4N was prepared by reacting a solution of AQ4N with anhydrous HCl gas (WO 00/05194). Use of the reagent HCl gas makes scale up of this reaction step difficult. If a salt of AQ4N is required, then this may be prepared by reacting a solution of AQ4N with an acid dissolved in a solvent. The reaction of AQ4N with an acid is preferably carried out at a reaction temperature of between −20° C. and −11° C. The acid can be added to the reaction dissolved in a solvent or can be generated in the reaction solution in-situ by the addition of the appropriate reagents. For example, hydrochloric acid may be generated in-situ by adding the reagents acetyl chloride and ethanol to the reaction solution.

Suitable inorganic acids include hydrochloric acid, hydrobromic acid, phosphoric acid and sulphuric acid. Suitable organic acids include acetic acid, dichloroacetic acid, malonic acid, maleic acid, tartaric acid, pimelic acid, lactic acid, citric acid and benzenesulfonic acid.

Suitable solvents for the formation of salts of AQ4N include dichloromethane, chloroform, dichloroethanes, carbon tetrachloride, toluene, 1,2-propanediol or a solvent mixture of any combination of these solvents. All these solvents can also be used as a mixture with or without aliphatic alkyl alcohols. The reaction solvent is preferably 1,2-propanediol or more preferably a solvent mix of dichloromethane and an aliphatic alkyl alcohol.

Purification of AQ4N or Its Salts

Previous literature processes were not effective at removing key impurities from salts of AQ4N. In another aspect of the present invention, it is preferable that one of the purification steps involving AQ4N or a salt of AQ4N is passing a solution of the compound through activated charcoal. This is an unusual method of purifying a coloured compound as treatment with charcoal is well known to the art as a method for removing coloured impurities. The final product obtained from this procedure does not have to be recrystallised, as in the method described in J. Chem. Soc., Perkin Trans. I, 1999, 2755.

Suitable solvents for this step include dichloromethane, chloroform, dichloroethanes, carbon tetrachloride, toluene, 1,2-propanediol or a solvent mixture of any combination of these solvents. All these solvents can also be used as a mixture with or without aliphatic alkyl alcohols. The reaction solvent is preferably 1,2-propanediol or more preferably a solvent mix of dichloromethane and an aliphatic alkyl alcohol. Typically the compound to be purified is in the solvent in which it was produced

Friedel-Crafts Acylation

The Friedel-Crafts acylation of p-hydroquinone in WO 00/05194 was performed by reaction between the solid reagents at 220° C. A high temperature and solid phase reaction conditions make this step difficult to perform on an industrial scale. A large quantity of gas is evolved during this reaction step.

Another aspect of the present invention is to perform this reaction step in a solvent at temperature not exceeding 200° C. Use of a solvent enables the reaction to be stirred, which in turn, means that lower reaction temperatures can be used. Lower temperatures reduce the rate of gas evolution from the reaction.

Suitable solvents for use in the Friedel-Crafts acylation of p-hydroquinone are 1,1,1,2-tetrachloroethane, 1,1,2,2-tetrachloroethane, nitrobenzene, chlorobenzene, 1,2-dichlorobenzene, toluene or a sulfone. These solvents are used independently or can be used in any combination with one another. The sulfone is preferably tetramethylene sulfone. The reaction is preferably conducted at a temperature not exceeding 180° C. with stirring of the reaction mixture.

After completion of the Friedel-Crafts reaction step, removal of the aluminium containing bi-product from the crude DDA is problematic, particularly when the reaction is carried out in the solid phase. Reduction of the aluminium content is important at this stage because it reduces the number of purification cycles in later stages of the process. This is an important consideration because potential exposure to the subsequent cytotoxic products, such as AQ4, can be minimised.

After carrying out the Friedel-Crafts reaction as described above, it is preferred that the aluminium containing bi-product contaminating the crude DDA is completely removed or reduced in quantity.

A preferred method for reducing the quantity of the aluminium containing bi-product is the formation of a slurry with the reaction solution by adding an acid. Reduction of the aluminium content in the crude DDA is achieved by slurrying the reaction solution several times, preferably with aqueous hydrochloric acid.

Another preferred method, that can be used independently or in conjunction with the slurrying procedure, is the addition of a chelating agent to the reaction solution. The chelating agent forms an aluminium complex with the aluminium containing impurities, which facilitates removal. Selection of an appropriate chelating agent allows the removal of the aluminium complex by precipitation from solution, use of phase transfer conditions or by using other size exclusion or filtration techniques.

Formation of AQ4

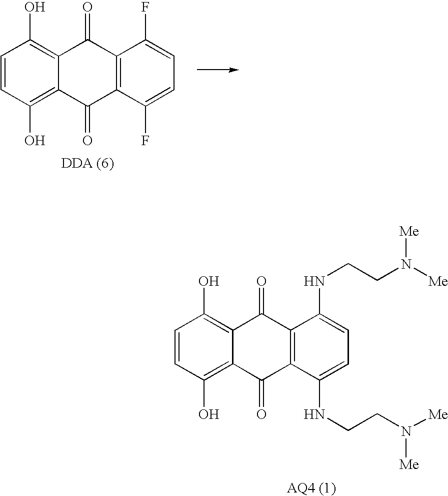

The synthesis of AQ4 from DDA described in WO 00/05194A produces moderate yields of product (˜40%). This reaction step is performed at a temperature ranging from 0° C. to 100° C. Suitable solvents for performing this reaction are tetrahydrofuran, collidine, lutidine, dimethylformamide, dimethylacetamide, N-methylpyrrolidone, dimethyl sulphoxide, diglyme and sulfolane. These solvents may also be used in any combination with one another.

The present invention concerns the isolation of AQ4 from the acidic bi-products that result in this reaction step. Neutralisation of the acidic bi-products is carried out with a suitable base. Suitable bases are dimethyl aminopyridine, N-methyl piperidine, N-methyl pyrrolidine, any tertiary amines, any water soluble tertiary amine or any of these bases that are attached to a solid phase support, group 1 alkali metal carbonates and bicarbonates. Preferably, a cooled aqueous ammonium hydroxide/brine solution is used to neutralise the acidic bi-products of the reaction. Preferably the workup of AQ4 is conducted at a temperature ranging from 10° C. to 30° C. This method results in improved yields of AQ4, even when the reaction is performed on a larger scale.

As will be appreciated by one of skill in the art, features and preferred embodiments of one aspect of the invention will also pertain to other aspects of the invention.

Definitions

The term pharmaceutically pure as used herein, pertains to a compound that is sufficiently pure for use as a pharmaceutical.

The term oxaziridine as used herein, pertains to a compound with a functional group that contains a saturated, three membered heterocylic ring formed from C, N and O, i.e.

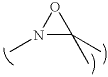

Particularly suitable oxaziridines include 2-benzene-sulfonyl-3-phenyl-oxaziridine, and those shown below.

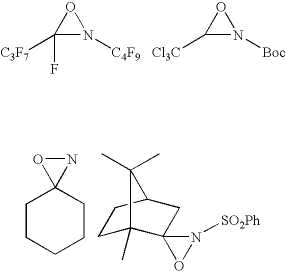

The term peracid as used herein, pertains to compounds that contain the —C(═O)OOH functional group. Particularly suitable peracids include m-chloroperbenzoic acid, peracetic acid, perbenzoic acid, trifluoroperacetic acid and 3,5-dinitroperoxybenzoic acid.

The term salt of a peracid as used herein, pertains to compounds that contain the anionic —C(═O)OO⁻ functional group with a suitable cation as defined below. Particularly suitable salts of peracids include magnesium monoperoxyphthalate, sodium peracetate and zinc peracetate.

The term aliphatic alkyl alcohol as used herein, pertains to compounds of the form R—OH, where R is a saturated linear or branched alkyl group. The term alkyl as used herein, pertains to a monovalent moiety obtained by removing a hydrogen atom from a carbon atom of a hydrocarbon compound having from 1 to 7 carbon atoms, which is saturated. Examples of saturated linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇). Examples of saturated branched alkyl groups include iso-propyl (C₃), iso-butyl (C₄), sec-butyl (C₄), tert-butyl (C₄), iso-pentyl (C₅), and neo-pentyl (C₅). Particularly suitable aliphatic alkyl alcohols include methanol, ethanol, propanol and propan-2-ol.

The term sulfone as used herein, pertains to a compound containing the C—S(═O)₂—C functional group. A particularly suitable sulfone is tetramethylene sulfone.

The term chelating agent as used herein, pertains to a compound that bonds from more than one position to a metal. The bonding atoms in the chelating compound can form part of a linear compound or part of a cyclic structure. Examples of chelating agents are ethylenediaminetetraacetic acid (EDTA), ethylenediamine (EDA) and diethylenetriamine (DETA), and their anions thereof. Examples of cyclic chelating agents are crown ethers, such as 18-crown-6 or 15-crown-5, crytands, spherands or porphyrins.

Unless otherwise specified, included in the above are the well known ionic, salt, solvate, and protected forms of these compounds.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

The term “prodrug”, as used herein, pertains to a compound which, when metabolised (e.g., in vivo), yields the desired active compound. Typically, the prodrug is inactive, or less active than the active compound, but may provide advantageous handling, administration, or metabolic properties.

Synthetic Details

Preparation of Crude DDA (6)

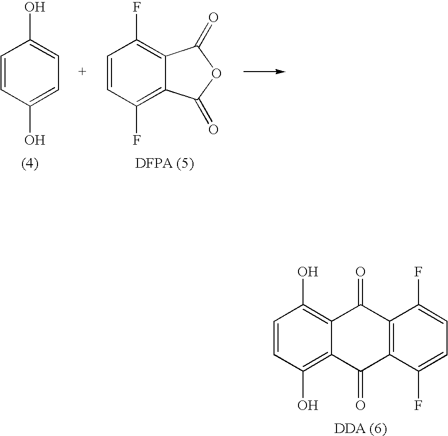

0.3 kg (1.63 mol) of DFPA, 0.2 kg (1.82 mol) of p-hydroquinone, 1.1 kg (8.25 mol) of powdered anhydrous AlCl₃ and tetramethylene sulfone (1.8 L) were stirred for approximately 12 hours at a temperature of 155° C. to 180° C. The reaction mixture was then quenched with ice cold water (1.0 L) and treated with aqueous 2M hydrochloric acid (1.0 L). The suspension was filtered and the resulting solid was dried at 45° C. under vacuum. A fine red powder (crude DDA) was obtained after drying. This reaction was performed on several batches of the same scale and yields of around 68% were obtained. The isolated DDA was contaminated with aluminium containing impurities, where the aluminium content in the isolated product ranged from 3290 ppm to 975 ppm, see Table 1.

All batches used 300 g as one molar equivalent of DDA with the exception of batch 5A, which used 200 g.

TABLE 1
Yield and purity of crude DDA

| Batch | Yield (g) | Analytical data |
|---|---|---|
| Number | (% yield) | Al (ppm) |
| 1A | 302 (67%) | 975 |
| 2A | 296 (66%) | 1150 |
| 3A | 305 (68%) | 3290 |
| 4A | 310 (69%) | 1165 |
| 5A | 256 (85%) | 1640 |

The organic components of the product were determined to be pure by using HPLC.

Purification of Crude DDA

The solid, crude DDA, obtained using the previous method, was treated with aqueous 2M hydrochloric acid (1.0 L) to form a slurry. Filtration of the suspension resulted in the isolation of a solid. The solid was slurried and filtered several times before drying the solid at 45° C. under vacuum to leave a fine red powder. The mass recovery for this procedure was typically 90%. This procedure resulted in a reduction of the aluminium content in the crude DDA for all the batches, see Table 2.

TABLE 2
Yield and purity after slurrying crude DDA

| Batch | Yield (g) | Analytical data |
|---|---|---|
| Number | (% recovery) | Al (ppm) |
| 1A | 283 (94%) | 714 |
| 2A | 276 (93%) | 801 |
| 3A | 268 (88%) | 875 |
| 4A | 280 (90%) | 865 |
| 5A | 229 (89%) | 526 |

The organic components of the product were determined to be pure by using HPLC.

Preparation and Purification of AQ4

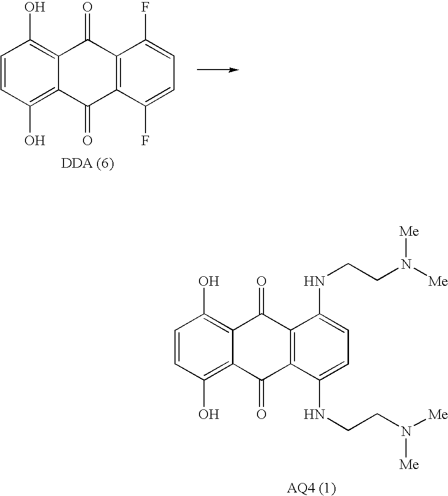

(AQ4 was prepared by a modification of the method listed in WO 00/05194). 0.5 kg (1.81 mol) of the DDA, obtained from the previous step, 1.3 L (12.08 mol) N,N-dimethylethylene diamine and pyridine (3.6 L) were stirred at 40° C. for 22 hours under a nitrogen atmosphere. A 2 L solution of 30% aqueous ammonium hydroxide/23% brine cooled to 0° C. was added to workup the reaction mixture. The resulting slurry was stirred at 0° C. for 3 to 4 hours and before isolating the blue solid product by filtration. The solid was washed with a 10% aqueous ammonium hydroxide solution (1.0 L) and then dried to a constant weight at 40° C. to 50° C. under vacuum. This procedure routinely yielded around 540 g (˜73%) of AQ4.

The purified DDA obtained from above was combined and then divided into several 500 g batches for use in this step.

TABLE 3
Yield and purity of AQ4

| Batch | Yield (g) | Analytical data |
|---|---|---|
| Number | (% yield) | Al (ppm) |
| 1B | 543 (73%) | 560 |
| 2B | 554 (74%) | 546 |
| 3B | 560 (75%) | 638 |
| 4B | 529 (71%) | 510 |

The purity of the product was verified using HPLC.

Synthesis of AQ4N

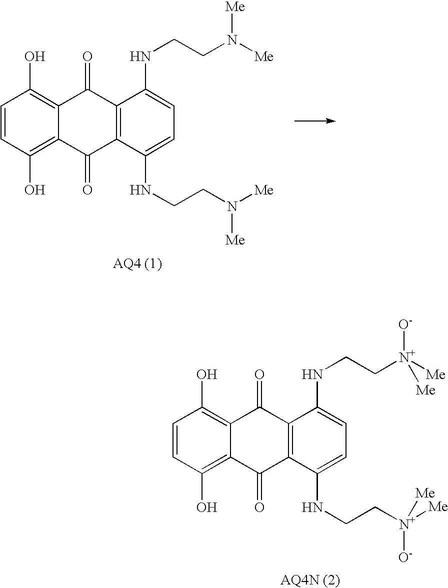

A solution containing 0.26 Kg (0.67 mol) of magnesium monoperoxyphthalate in methanol (0.5 L) was added dropwise to a stirred solution of 0.2 kg (0.48 mol) of AQ4 from the previous step, methanol (0.8 L) and dichloromethane (2.9 L) cooled to approximately −11° C. After the addition was complete, the reaction solution was allowed to warm to 0° C. and was then stirred for 18 hours. The AQ4N can then be isolated at this stage using literature procedures.

Preparation and Purification of the Dihydrochloride Salt of AQ4N (AQ4N.2HCl)

Ethyl acetate (4.0 L) and ethanol (0.8 L) were added to a solution of AQ4N, such as the reaction solution from the previous step, so as to maintain the solution temperature of 0° C. After agitating at this temperature for 1 hour, the stirred solution was then cooled to approximately −11° C. and 0.2 L (2.8 mol) of acetyl chloride was added dropwise. The resulting slurry was then stirred for 10 min at this temperature before quick filtration of the reaction mixture. A crude solid was obtained, which was washed with a mixture of ethanol and water (2 L), and then dried under vacuum until the weight was constant. 220 g (˜88%) of reasonably pure AQ4N.2HCl was typically obtained, see Table 4. HPLC was used to check the purity of the organic content of the product.

TABLE 4
Yield and purity of crude AQ4N•2HCl

| Batch | Yield (g) | Analytical data |
|---|---|---|
| Number | (% yield) | Purity (% area) |
| 1C | 217 (86%) | 95 |
| 2C | 235 (94%) | 95 |
| 3C | 196 (78%) | 95 |
| 4C | 225 (90%) | 95 |

For pharmaceutical purity, 100 g of the product obtained above was subjected to an additional purification step. The crude AQ4N.2HCl was dissolved in water (0.6 L) and treated with activated charcoal (0.06 Kg). Charcoal was removed from the resulting suspension by filtration. The product was precipitated from the mother liquors by ethanol addition (1.4 L) and cooling the solution to 0° C. The precipitate was isolated by filtration and then washed with ethanol (4.0 L). The AQ4N.2HCl product was dried under vacuum at ambient temperature. The mass recovery for this procedure was around 60%.

TABLE 5
Purification of AQ4N•2HCl

| Batch | Yield (g) | Analytical data |
|---|---|---|
| Number | (% recovery) | Purity (% area) |
| 1D | 61.7 (61.7%) | 97 |
| 2D | 62 (62.0%) | 97 |

HPLC showed that purity of the organic content of the product had increased.

1. A process for the preparation of compound AQ4N of formula (2): 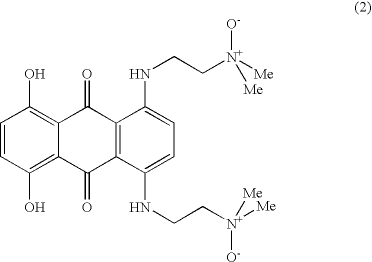
or a salt or solvate thereof wherein the said process includes the reaction step: 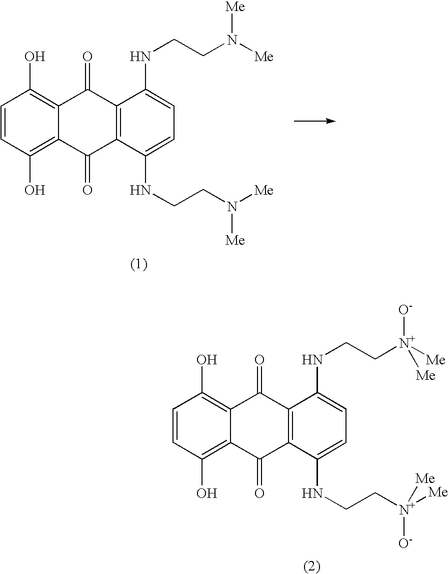
where compound AQ4 of formula (1) is oxidised to compound AQ4N of formula (2) with an oxidising agent at a reaction temperature not exceeding 10° C., where the oxidising agent is a peracid or salt of a peracid, and where the oxidising agent is added at a temperature not exceeding 0° C.

2. A process according to claim 1 where the oxidising agent is magnesium monoperoxyphthalate.

3. A process according to claim 1 where the reaction is conducted at a temperature not exceeding 0° C.

4. A process according to claim 1 to where the reaction solvent is 1,2-propanediol, dichloromethane or an aliphatic alkyl alcohol.

5. A process according to claim 1 for the preparation of a salt of AQ4N, where the salt of AQ4N, or a solvate thereof, is prepared by reaction of compound AQ4N of formula (2) with a solution of hydrogen chloride.

6. A process according to claim 1 where a solution containing AQ4N or a salt of AQ4N is treated with activated charcoal.

7. A process for the preparation of compound AQ4N of formula (2) 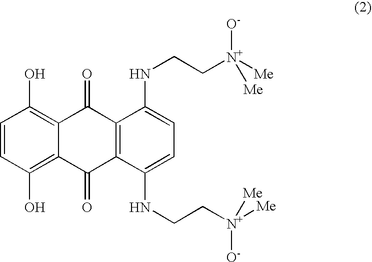
that includes the reaction step: 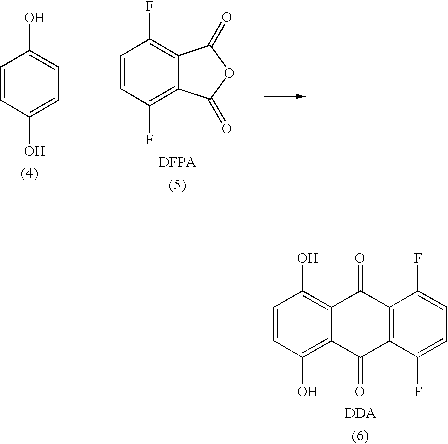
wherein the said reaction step is conducted in a stirrable solvent at a temperature not exceeding 200° C.

8. A process according to claim 7 wherein the solvent is tetramethylene sulfone.

9. A process according to claim 7 where the crude compound DDA of formula (6) is treated by slurrying several times with aqueous hydrochloric acid.

10. A process according to claim 7 where the crude compound DDA of formula (6) is treated by adding a chelating agent.

11. A process for the preparation of compound AQ4N of formula (2) 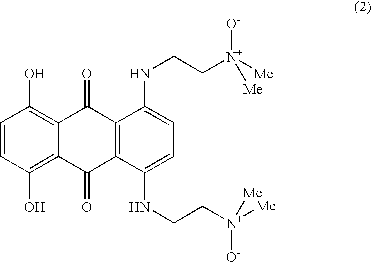
which includes the reaction step: 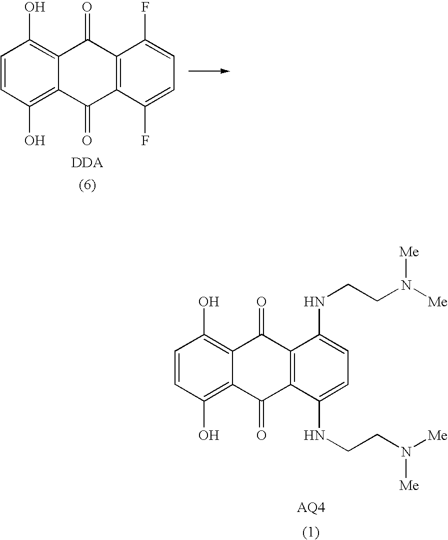
wherein the reaction solution of the said reaction step is treated with an ammonium hydroxide and brine solution cooled to 0° C.
